(19) United States
(12) Patent Application Publication
(10) Pub. No.: US 2004/0116329 A1
(43) Pub. Date: Jun. 17, 2004

(54) Inhibition of proteasomes to prevent restenosis

(76) Inventor: Stephen E. Epstein, Rockville, MD (US)

Correspondence Address: Heller Ehrman, White & McAuliffe, Suite 300, 1666 K Street NW, Washington, DC, 20006, US

(21) Appl. No.: 10/470,533

(22) PCT Filed: Jan. 29, 2002
(86) PCT No.: PCT/US02/02367

(51) Int. Cl. A61K 38/00

(52) U.S. Cl. 514/2

(57) ABSTRACT

Restenosis of blood vessels after angioplasty is achieved by preventing of cell proliferation, particularly of smooth muscle cells, in blood vessel walls by inhibiting the ubiquitin-proteasome protein degradation pathway. The inhibition is accomplished by administering to the cells in the blood vessel walls a compound, e.g., a protein or small molecule, capable of inhibiting the ubiquitin-proteasome protein degradation pathway. The inhibiting compound is preferably administered by coating the compound on a stent and implanting the stent in the blood vessel after angioplasty.

### RELATIONSHIP TO OTHER APPLICATIONS

This application claims the benefit of copending U.S. Provisional Patent Application No. 60/264,735, filed Jan. 30, 2001.

### BACKGROUND OF THE INVENTION

1. Field of the Invention

This invention relates to methods of preventing restenosis after angioplasty and more particularly to methods of preventing restenosis by applying a stent coated with a material capable of inhibiting the action of cellular proteasomes.

2. Brief Description of the Prior Art

Surgical intervention by means of balloon angioplasty or bypass grafting has become a common strategy for alleviating stenosis of cardiac arteries that have become narrowed or obstructed by accumulation of atheromatous plaque.

Unfortunately, although the initial success rate of coronary angioplasty for opening obstructed coronary arteries exceeds 95%, restenosis occurs at the site of angioplasty in 25-50% of patients within six months, regardless of the type of angioplasty procedure used.

Two primary mechanisms appear to be involved in the development of restenosis. First, recoil of the vessel wall (negative remodeling) leads to gradual narrowing of the vessel lumen. Second, an exaggerated healing response of medial and/or adventitial smooth muscle cells (SMCs) to vascular injury takes place, which involves the excessive proliferation of SMCs and the migration of SMCs to the subintima, where they continue to proliferate and begin to secrete extracellular matrix. These processes involving SMCs cause the neointimal mass to expand and gradually encroach upon the coronary lumen; ultimately the expanding lesion narrows the vessel, increases resistance to blood flow, and causes ischemic symptoms. In the absence of stenting, both remodeling and an expanding neointima contribute to restenosis. When stents are deployed, negative vascular remodeling is prevented and restenosis occurs only as a result of the expanding neointimal mass.

Increasing information about the molecular and cellular mechanisms involved in the SMC response to vascular injury including SMC proliferation and migration, has led to new molecular targets to prevent restenosis. For example, it has been learned that the proliferation of the SMCs within the cell wall can be inhibited by interfering with the molecular mechanism by which the proliferation of the SMCs is controlled, or by affecting the microtubular system within the cell to discourage proliferation.

Proteins Controlling Progress of Cells Through the Cell-Cycle:

SMCs within the vessel wall are normally in a quiescent state. Immediately after injury, however, early response genes are expressed and the cells enter the cell cycle, wherein their replication is tightly regulated by an array of cell cycle regulatory proteins acting conjointly and in sequence at various points of the cycle. These regulatory proteins include cyclin-dependent kinases (cdc2 and cdk2), which phosphorylate critical regulatory proteins, and the interaction of such kinases with cyclin-dependent kinase inhibitors, such as p16, p21, and p27kipl. Changes in the levels of these inhibitors exert marked effects on cell-cycle progression, through inhibition of critical phosphorylation reactions.

One of the proteins involved in cell cycle progression that is regulated by phosphorylation is the tumor suppressor protein retinoblastoma protein (pRb). In the hypophosphorylated state pRb complexes with DNA binding and gene activating proteins, such as E2F, thereby exerting an inhibiting effect on cell cycle progression in G₀/mid G₁. Upon phosphorylation, the pRb/E2F complex dissociates, freeing E2F to bind to its DNA binding sites and consequently stimulate the transcription of genes inducing progression to the S phase of the cell cycle.

Microtubular Modulation of the Response of Cells to Mitogens and Cytokines:

The microtubular system has been shown to modulate the response of cells to various mitogens and cytokines through activation of transmembrane signalling cascades. Targets of these pathways include activation of kinases, including mitogen-activated protein kinase activity, changes associated with microtubular depolymerization. The microtubules have also shown to play a part in the changes in SMCs that lead to their contributing to the restenosis lesion.

Therapeutic Attempts to Inhibit Restenosis:

Many attempts have been made to prevent the development of restenosis. Although many have been reported to be successful in inhibiting neointima development in various experimental models, with the notable exception of brachytherapy, their translation to clinical interventions has uniformly been without success.

Recently, however, the results from two drugs have been encouraging: paclitaxel (Taxol®) and rapamycin. The success of these drugs is based on the cellular and molecular effects on the two processes described above: microtubular modulation of the response of cells to mitogens and cytokines, and proteins controlling progress of cells through the cell-cycle.

Microtubular Modulation of the Response of Cells to Mitogens: Paclitaxel:

Paclitaxel favors stabilization of microtubule assembly, forming numerous disorganized microtubules within the cytoplasm, and thereby inhibiting many of the microtubular mediated cell signaling cascades cited above, including inhibition of cell division, predominantly in the G₀/G₁ and G₂/M phases of the cell cycle. Importantly, paclitaxel in biologically relevant concentrations does not appear to induce apoptosis. Taxol® inhibited, in vitro, both platelet-derived growth factor-stimulated SMC migration and SMC proliferation, and in vivo, inhibited neointimal accumulation in the rat carotid artery injury model.

Proteins Controlling Progress of Cells Through the Cell-Cycle:

Rapamycin, a macrolide antibiotic, is a potent inhibitor of cell proliferation. It has recently been shown in a pig coronary artery injury model to significantly reduce the neointimal response to injury. The mechanism of action of rapacmycin almost certainly largely derives from its ability to interfere with cell cycling. Thus, downregulation of p27kip by mitogens is blocked by rapamycin; consistent with this activity, in the porcine injury model cited above, rapamycin administration was associated with increased p27 levels and inhibition of pRb phosphorylation within the vessel wall. The most likely relevant molecular mechanisms are as follows; after binding to its cytosolic receptor, FKBPl2, rapamycin increases p27, reduces cdc2 and cdk2 activity, and inhibits pRb phosphorylation, thereby releasing E2F which leads to a broad array of transcription of genes leading to cell cycle progression.

In order to assure a satisfactory therapeutic response, the active ingredient must be provided to the cells to be affected in a sufficiently high concentration. A number of strategies for drug delivery have been tried, for example, catheter delivery and coated stents.

Catheter delivery systems: Therapeutic strategies began to focus on local delivery, as it became apparent that high concentrations of active agent were needed at the target site. It would be very unlikely that such high concentrations could be achieved by any other approach than local delivery.

Unfortunately, despite years of development and testing, the consensus is that catheter delivery systems are too inefficient to be successful—only one percent or less of the delivered product appears to persist for any period of time in the vessel wall.

Coated stents to deliver proteins or small molecules: The concept that drugs could be incorporated into the coating of stents has become popularized, with mixed results. Most studies have shown no effect, but preliminary encouraging results of stents in which their coating is impregnated with either paclitaxel or rapamycin, or their derivatives, have been reported at several international meetings.

Although either or both of the above-described strategies, using paclitaxel or rapamycin (or their congeners), may prove to be efficacious, the history of the search for anti-restenosis treatment over the years suggests that no one form of therapy will prove to be efficacious, and at the same time free of important side effects. It also seems reasonable to consider that combinations of therapies, targeting different mechanisms involved in the restenosis process, will be more effective than the use of any single agent. Such a multiple targeting approach might also improve safety, as lower doses of each therapeutic agent with different target mechanisms could be more effective at a lower dose. And since their side-effect profiles will probably be different, the lower doses needed to achieve efficacy would be expected to result in a lower incidence of side effects.

Accordingly, a need has continued to exist for additional methods of inhibiting restenosis.

### SUMMARY OF THE INVENTION

This need has been addressed by the strategy of this invention wherein the inhibition of restenosis is achieved by inhibiting a new target within the cells responsible for the stenotic proliferation of tissue in order to prevent the multiple processes involved in restenosis.

According to the invention inhibition of cell proliferation, particularly of smooth muscle cells in arterial walls is achieved by inhibiting the ubiquitin-proteasome protein degradation pathway, thereby interfering with proliferation of cells that could contribute to restenosis.

Accordingly, it is an object of the invention to provide a method for preventing restenosis of a blood vessel after an angioplastic procedure.

A further object is to provide a method for inhibiting the ubiquitin-proteasome protein degradation pathway in cells capable of proliferating to cause restenosis.

A further object is to provide a method for local delivery of an effective concentration of a drug capable of inhibiting the ubiquitin-proteasome protein degradation pathway in cells capable of proliferating to cause restenosis.

A further object is to provide an intravascular stent coated with a compound capable of inhibiting the ubiquitin-proteasome protein degradation pathway in cells capable of proliferating to cause restenosis.

Further objects of the invention will become apparent from the disclosure of the invention which follows.

### DETAILED DESCRIPTION OF THE INVENTION AND PREFERRED EMBODIMENTS

One mechanism utilized by cells to alter the intensity of signaling conducted along specific signaling cascades is to regulate levels of the signal transduction proteins participating in particular pathways. In this regard, the ubiquitin-proteasome pathway is responsible for the degradation of multiple proteins. Specific proteins targeted for degradation by this pathway are identified as targets by undergoing ubiquitinization. Proteasomes, which are large complexes of proteolytic enzymes, recognize these ubiquitin molecular tags and initiate the process of intracellular degradation. By degrading signaling proteins, the proteasome is involved in altering many cellular signals, including those involved in growth and differentiation.

For example, p53 is a suppressor gene with multiple critical cellular functions, including the activation of a p53-modulated apoptosis pathway, and p53-mediated inhibition of cell cycle progression. In normal cells a protein, called MDM2, binds to the p53 protein, and in so doing, enhances the transfer of ubiquitin to p53. This maintains p53 at low levels by increasing proteasome-mediated p53 degradation. Inhibition of this pathway would result in elevated levels of p53, which would thereby predispose to apoptosis and inhibit cell cycle progression.

Another example is based on the effects inhibition of the ubiquitin-proteasome degradation pathway has on stabilizing cell cycle regulatory proteins and thereby interfering with cell proliferation. Thus, IFNγ inhibits proliferation of certain cell types. Data from a recent study suggests that this effect is caused, at least in part, by an IFNγ-induced inhibition of ubiquitin-proteasome-mediated degradation of p21, a potent inhibitor of cell cycle progression.

Recently, Millenium Pharmaceuticals presented research results of preclinical and clinical studies of LDP-341, which was described as a proteasome inhibitor and the first compound in a new class of chemotherapeutic agents. The data was presented at the 11th NCI-EORTC-AACR (National Cancer Institute, European Organization for Research and Treatment of Cancer, American Association for Cancer Research) Symposium on New Drugs in Cancer Therapy in Amsterdam. LDP-341 has been found to induce apoptosis through the stabilization of proteins critical for cell cycle progression and control of apoptosis. The drug was found to be effective in reducing the volume of various tumors. Although the drug was described as an anti-cancer agent, its activities suggest that LDP-341 will also exhibit potent anti-restenosis efficacy.

Interestingly, it has been described by Millenium that LDP-341 appears very active in tumor treatment when given in combination with standard chemotherapeutic agents or radiation. Because restenosis is treated by chemotherapeutic agents and by radiation, it is believed that LDP-341 will have additive, or even synergistic, effects in preventing restenosis when given in conjunction with these agents.

The major side effects of this drug, given orally, were gastrointestinal in nature. It therefore is to be expected that this drug, when administered via a stent-based platform, by achieving high local concentrations at the vessel wall and low systemic concentrations, will be effective with minimal systemic side effects.

According to the invention, a protein or small molecule with ubiquitin-proteasome-inhibiting activity, or plasmid DNA or a viral vector encoding such a protein or small molecule, is incorporated into a stent coating. The stent coating comprises a substance that adheres to the stent, and which will incorporate the molecule or DNA or viral vector without damaging it. Accordingly, when the stent is implanted after an angioplastic procedure, the protein or small molecule is delivered directly to the cells within the injured vessel wall (or to the cells that are migrating from the media and/or adventitia to form the neointima). The implantation of a coated stent according to the invention can be performed in any artery or interposed vein (such as, but not limited to, a saphenous vein graft to a coronary artery) that is obstructed and thereby impairs blood flow to the target tissue (whether it be heart or leg). In the case of plasmid DNA or a viral vector encoding such a molecule, the stent platform will facilitate transduction of cells within the vessel wall (or transduction of cells that are migrating from the media and/or adventitia to form the neointima) with the gene encoding the inhibitory molecule. The invention will employ any coating that can be attached to a stent and that has the above characteristics, and any molecule with ubiquitin-proteasome-inhibiting activity, or gene that encodes such a molecule.

The therapuetic concept on which the invention is based is as follows. The intimate and prolonged contact between the ubiquitin-proteasome-inhibiting activity of the protein or small molecule (or the gene encoding such a protein) that is contained within the stent coating, and vessel wall cells, will lead to high levels of ubiquitin-proteasome-inhibiting activity. This will exert the desired therapeutic effects on these cells, such as (but not limited to) inhibition of smooth muscle cell (SMC) proliferation or migration, and induction of SMC apoptosis.

Thus, the invention includes 1) the delivery system consisting of a stent coating that can be impregnated with the ubiquitin-proteasome-inhibiting protein, small molecule, or gene encoding such a product, and 2) the ubiquitin-proteasome-inhibiting protein, small molecule, or gene encoding such a product. Thus, the invention will have the benefits of substantially reducing the incidence of restenosis with minimal incidence of untoward complications, a result that has been achieved to only a limited extent (or, as with radiation therapy, carrying unknown future risk) with other anti-restenosis strategies. The method of the invention can also provide synergistic effects when applied with interventions targeting different molecular mechanisms involved in restenosis.

The therapeutic agents used in this invention can be any ubiquitin-proteasome-inhibiting protein, small molecule, or gene, e.g., plasmid DNA or viral vector. An example of a suitable therapeutic agent is LDP-341 of Millenium Pharmaceuticals, Inc. However, any compound, protein or small molecule, that inhibits the ubiquitin-proteasome protein degradation pathway, or any gene that codes for such a compound is suitable use in the method of the invention.

The coated stent used to administer the ubiquitin-proteasome inhibitor to the wall of the blood vessel after angioplasty may be any conventional stent suitable for supporting the vessel wall and maintaining the lumen thereof as is conventional in angioplastic procedures. The ubiquitin-proteasome inhibitor is coated onto the surface of the stent by incorporation into a coating that is adherent to the stent surface. Any stent coating that has the proper release kinetics for the ubiquitin-proteasome-inhibiting protein, small molecule, or gene encoding such a product, and that allows for viable incorporation of such a molecule, and that allows for such product to reside in the coating for days or weeks, will be appropriate. Incorporation of such a molecule into the stent would be based on well-known technology for those expert in the field of stent-based drug delivery. A suitable coating is that prepared by the Photolink® process of theSurModics company (Eden Prairie, Minn.).

The invention having now been fully described, it should be understood that it may be embodied in other specific forms or variations without departing from its spirit or essential characteristics. Accordingly, the embodiments described above are to be considered in all respects as illustrative and not restrictive, the scope of the invention being indicated by the appended claims rather than the foregoing description, and all changes which come within the meaning and range of equivalency of the claims are intended to be embraced therein.

I claim:

1. A method of preventing cell proliferation in blood vessel walls after angioplasty comprising administering to at least one cell in a blood vessel wall after an angioplastic procedure an amount of a compound capable of inhibiting the ubiquitin-proteasome protein degradation pathway in said cell effective to prevent proliferation of said cell.

2. The method of claim 1 wherein said compound is LDP-341.

3. The method of claim 1 wherein said compound is administered by coating said compound on a stent and implanting said stent within said blood vessel.

4. The method of claim 3 wherein said compound is LDP-341.

5. An intravascular stent coated with a compound capable of inhibiting the ubiquitin-proteasome protein degradation pathway in a cell.

6. The stent of claim 5 wherein said compound is LDP-341.
